(19) United States
(12) Patent Application Publication
(10) Pub. No.: US 2004/0015112 A1
(43) Pub. Date: Jan. 22, 2004

(54) Controlled motion ankle walker brace

(76) Inventors: E. Gerald Salutterback, Miramar, FL (US); Rhonda Machin, Weston, FL (US); Jack Lentz, West Miami, FL (US)

Correspondence Address: DOUGHERTY, CLEMENTS & HOFER, 1901 ROXBOROUGH ROAD, SUITE300, CHARLOTTE, NC, 28211, US

(21) Appl. No.: 10/134,083

(22) Filed: Apr. 26, 2002

Related U.S. Application Data
(63) Continuation-in-part of application No. 29/155,655, filed on Feb. 14, 2002.

(51) Int. Cl. A61F 5/00

(52) U.S. Cl. 602/22

(57) ABSTRACT

A controlled motion ankle walker brace for facilitating the rehabilitation process of an injured ankle and other injuries to the ankle region of a patient. The device includes a liner which wraps around the lower leg, ankle and foot of the patient to snugly and cushioningly hold the foot, a rocker-type shoe into which the patient's foot is removably secured, and a hinge having a lower strut attached to the shoe and an upper strut extending along the lower leg of the patient. The upper strut has an enlarged axle to which the lower strut is rotatably attached such that the upper strut and lower strut rotate in relation to each other. A channel is formed in the lower portion of the upper strut and contiguously extends into the lower strut for receiving a chock. A variety of chocks, corresponding to various degrees of rotation are insertable into the channel to set the range of motion for the ankle walker brace in dorsiflexion and plantar flexion. The range of motion of the ankle walker brace is accurately and quickly set by the patient by simply attaching the desired chock into the channel by a single fastener. The configuration of the hinge allows for its construction to be of lightweight injection molded plastic material to aid the patient in having a more normal gait.

### CROSS REFERENCE TO RELATED APPLICATION

This application is a Continuation-In-Part of U.S. Design application Ser. No. 29/155,655, filed Feb. 14, 2002.

### FIELD OF THE INVENTION

The present invention relates to a method and apparatus for controlling a range of motion of an ankle joint, and more particularly to an orthotic device having selectable controlled ankle motion for decreasing rehabilitation time for injuries to an ankle joint.

### BACKGROUND OF THE INVENTION

Ankle walkers have been used for treatment of Achilles tendon repair, fractures of the lower leg and ankle, acute ankle sprains, and soft tissue injury. The configurations of ankle walkers permit patients to closely simulate a natural walking gait while the affected ankle joint is stabilized within a predetermined range of motion in dorsiflexion and plantar flexion. As a result, the movement of a patient's ankle is controlled over a fixed range of flexing while weight can be applied to the affected area during walking.

Traditionally, a fracture or other severe injury to the ankle was treated by holding the foot in a neutral position and immobilizing the ankle in a rigid cast from below the knee to the toes. After six to eight weeks a walking boot cast was applied and weight bearing permitted. The walking boot cast also mobilized the ankle joint. The cast was uncomfortable due to its weight and was not removable during its long period of use. Additionally, the rigidness of the cast limited the mobility of the patient.

During the 1970's, rigid ankle braces referred to as “walkers” were developed for replacing the plaster walking cast during healing and rehabilitation of ankle fractures and injuries. Ankle walkers have many advantages over the rigid walking boot cast. For instance, ankle walkers save the time normally required to apply a plaster cast, and cast complications are avoided. Ankle walkers can be applied with an adjustable snug fit, and can be easily removed which provides convenient access to the afflicted joint. Since the patient can come out of the cast earlier and begin walking, healing is improved and the rehabilitation period is shortened as a result of weight-bearing while walking. Weight-bearing decreases atrophy, maintains propricepion, and reduces edema.

During the 1990's, ankle walkers experienced many advances and, accordingly, their usage grew dramatically. Currently, most ankle walkers include a foot supporting shoe, referred to as a “rocker”, which includes a rigid shell that fits around and under the foot and a curved sole on the bottom of the shell to permit a rocking heel-to-toe motion of the foot during use. Struts extend from the rocker along the lateral and medial sides of the leg to above the ankle joint. The struts are provided with a hinge which can be set to immobilize the patient's ankle joint or allow the ankle joint to rotate through a fixed angle. These struts are commonly affixed to a liner which is wrapped around the patient's leg, ankle and foot, by straps.

Prior art ankle walkers utilize aluminum or steel hinge assemblies to control the degree of rotation of the ankle joint in plantar flexion and dorsiflexion. A common hinge configuration is a pin-style that includes several metal plates and is set by placing a pin in a series of set holes which correspond to the desired range of motion. This hinge is complicated by requiring several components, which makes assembly of the product difficult. Additionally, this configuration must be fabricated from aluminum or steel to ensure that the hinge can withstand forces applied to it during use. These materials add both cost and overall weight to the final product.

Another common configuration for an ankle walker hinge includes a pair of set screws which are adjusted up and down to set the degree of rotation for the ankle joint. A drawback of this hinge is that it is indefinitely adjustable and hard to set at an exact point. Accordingly, it is difficult to set both sides of the brace to the same setting which can cause the brace to malfunction and/or lead to injury of the patient. Additionally, this design must be fabricated from aluminum or steel which adds to the cost and weight of the overall product.

What is needed is a range of motion hinge for use in an ankle walker which distributes forces in such manner that it can be made from injection molded plastic composite material so that the ankle walker is light and cost effective to produce. Also needed is an ankle walker which has a minimal number of parts and is simple to accurately adjust the angle of rotation by the patient without assistance from a medical professional.

Applicant is aware of the following U.S. patents relating to orthotic ankle devices:

| U.S. Pat. No. | Issue Date | Inventor | TITLE |
|---|---|---|---|
| 6,155,998 | Dec. 5, 2000 | Gilmour | Walker |
| 5,997,493 | Dec. 7, 1999 | Young | Hinge With Movement |
| | | Limitation | |
| 5,885,235 | Mar. 23, 1999 | Opahle, et al. | Joint Brace And More |
| | | | Particularly A Knee |
| | | | Brace |
| 5,611,773 | Mar. 18, 1997 | Nash et al. | Range of Motion Cap |
| | | | for Range Of Motion |
| | | | Orthotic |
| 5,460,599 | Oct. 24, 1995 | Davis et al. | Orthopedic Hinge |
| | | | Assembly For A Leg |
| | | | Brace |
| 5,429,588 | Jul. 4, 1995 | Young et al. | Ankle Foot Orthoses |
| | | | Known As Lower Leg |
| | | | Walkers |
| 5,372,574 | Dec. 13, 1994 | Hino et al. | Artificial Limb Joint |
| | | | And Joint Device |
| 5,176,623 | Jan. 5, 1993 | Stetman et al. | Multiple Fixed Angle |
| | | | Orthopedic Appliance |
| 5,092,321 | Mar. 3, 1992 | Spademan | Walker Brace |
| 5,044,360 | Sept. 3, 1991 | Janke | Orthosis With Variable |
| | | | Motion Controls |
| 5,000,169 | Mar. 19, 1991 | Swicegood | Adjustable Flexion- |
| | | et al. | Extension Hinge For |
| | | | Hinged Limb |
| | | | Immobilizer |
| 4,962,760 | Oct. 16, 1990 | Jones | Orthopedic Restraint |
| | | | Apparatus |
| 4,738,252 | Apr. 19, 1988 | Friddle et al. | Mechanical Joint |
| | | | Construction |

Gilmore, U.S. Pat. No. 6,155,998, discloses a walker comprising a frame and a pair of arms. The frames carry a pair of protrusions of which each are provided with an aperture, which align with openings provided in the arms. An inner plate carries a shaft which extends through the apertures to provide a pivotal point for the walker. The inner plate is shaped to provide a pair of shoulders which rotate with the inner plate to strike against pins positioned in apertures. The range of motion in either the dorsi or plantar directions can be varied by suitably positioning the pins.

Young, U.S. Pat. No. 5,997,493, teaches a hinge for use in orthopedic brace comprising a first member rotatably mounted to a second member. The first member has at least one limiting means which is selectable and lockable in a variety of incremental positions by means of engaging teeth. The second member has a projection which prevents relative movement of the hinge members in a given direction once the projection contacts the limiting means.

Janke, U.S. Pat. No. 5,044,360, teaches a control motion ankle-foot orthosis comprising a shell releasably attachable to the lower leg, a foot support releasably attachable to the foot, and lateral and medial control motion ankle-joint assemblies attached to the shell and to the foot support and positioned in alignment with an axis of rotation through the ankle joint. Each ankle joint assembly include a rigid support plate affixed at one end to the shell, extending across the ankle joint, and affixed to the other end of the foot support. A cam member is secured to the foot support and rotatably mounted to the support plate to allow the shell and foot support to rotate in relation to each other. The support plate is provided with a forward and rearward stop against which the cam member engages to control angular motion in the plantar flexion and dorsiflexion. Cam members come in a variety of sizes that contact with corresponding interior and posterior portions of the stop to provide selected means of controlling different angles of rotation in the plantar flexion in dorsiflexion.

Jones, U.S. Pat. No. 4,962,670, teaches an orthopedic restraint apparatus having lower leg brace members, a foot support, and an adjustable ankle joint articulation system rotatably connecting the leg members to the foot support. The foot support includes a pair of riser members which moveably insert within a channel formed in the lower end of the upper brace member. Pins are inserted into the channel to restrict movement of the riser members. Accordingly, articulation of the upper brace member and either the dorsi or plantar direction is limited. Placement of the pins in a variety of locations allows for different degrees of articulation of the upper brace member.

### SUMMARY OF THE INVENTION

The present invention is a device for controlling the range of motion of an ankle joint to facilitate rehabilitation of injuries to that joint. During normal operation, the ankle walker is attached to the patient's lower leg and foot, and includes a pair of hinges positioned across the ankle of the patient to allow limited rotation of the ankle joint in plantar flexion and dorsiflexion. A variety of chocks, corresponding to various degrees of rotation, are interchangeably attachable to the hinges for setting a range of motion.

In the broadest sense, the control motion hinge for an ankle walker includes a hinge having a first strut and a second strut. The first strut includes a first end and a second end. The second strut includes a first end rotatably attached to the first strut second end so that the first strut is rotatable through an angle relative to the second strut about a pivot axis through a joint of the wearer. A channel has a first section, with forward and rearward abutment surfaces, is formed in the second end of the first strut, and a second section contiguous with the first section, having forward and rearward abutment surfaces, is formed in the second strut first end. A chock is removably attachable within the channel and includes a first portion and a second portion. The first portion includes forward and rearward shoulders positionable within the first channel section so that rotation of the first strut in dorsiflexion about the pivot axis causes of the first strut rearward abutment surface to engage with the first portion rearward shoulder to limit rotation of the first strut to a specific first angle, and so that rotation of the first strut in plantar flexion causes the first strut forward abutment surface to engage the first portion forward shoulder to limit rotation of the first strut to a specific second angle. The second chock portion includes forward and rearward shoulders positionable within the second channel section and is positioned adjacent to the second channel section abutment surfaces. Preferably, the first strut and the second strut are rotatably attached by an axle having a diameter of at least one inch. More preferably, the chock is removably affixed only to second strut.

In the broadest sense, the present invention also relates to a controlled motion ankle walker hinge comprising a hinge which includes a first strut and a second strut. The first strut has a first end for extending along the side of a wearer's leg, and a second end. The second strut has a first end rotatably attached to the first strut second end so that the first strut is rotatable through an angle relative to the second strut about a pivot axis, and a second end. A channel having a first section is formed in the first strut second end, and a second section contiguous with the first section is formed in the second strut first end. Means, releasably attachable within the channel, is provided for limiting rotation of the first strut in dorsiflexion and plantar flexion to respective specific angles. Means is also provided for releasably attaching the rotation limiting means to the hinge. Further means is provided for releasably attaching the ankle walker hinge to the second leg of the wearer. Preferably, the ankle walker hinge includes an axle having a diameter of at least one inch which rotatably secures the first strut to second strut.

### OBJECTS OF THE INVENTION

The principal object of the present invention is to provide a device for the rehabilitation of an ankle injury.

Another object of the invention is to provide apparatus which allows an injured ankle to be weight-bearing during rehabilitation.

A further object of this invention is to provide a device which allows the ankle joint to rotate over a pre-set range of motion in dorsiflexion and plantar flexion.

Another object of this invention to provide an ankle walker device which is lightweight.

Another object of the invention is to provide an ankle walker device which is made of injection molded plastic composite material.

Another object of this invention is to provide an ankle walker device which requires few parts and is simplistic in construction.

A further object of this invention is to provide an ankle walker device for which the angle of rotation in dorsiflexion and plantar flexion can be accurately set.

Another object of this invention is to provide an ankle walker device in which the degree of rotation in dorsiflexion and plantar flexion can be easily adjusted by a patient without assistance of a medical professional.

### BRIEF DESCRIPTION OF THE DRAWINGS

The foregoing and other objects will become more readily apparent by referring to the following detailed description and the appended drawings in which:

FIG. 1 is a front view of the invented controlled motion ankle walker having a hinge attached to a shoe and a sleeve for promoting rehabilitation of an injured ankle;

FIG. 2 is a front perspective view of the hinge of FIG. 1;

FIG. 3 is an exploded view of the hinge of FIG. 2;

FIG. 4 is a perspective view of the hinge of FIG. 2 showing a chock detached from the hinge;

FIG. 5 is a rear perspective view of the hinge of FIG. 2;

FIG. 6 is an exploded view of the hinge of FIG. 5;

FIG. 7 is a front view of several different chocks illustrating various combinations of anterior and posterior chock shoulders for selectively adjusting a range of motion of the ankle walker brace in dorsiflexion and plantar flexion about an axis through the ankle joint; and

FIG. 8 is a front view of an alternative controlled motion ankle walker brace for treating injury to the tibia or fibula.

### DETAILED DESCRIPTION

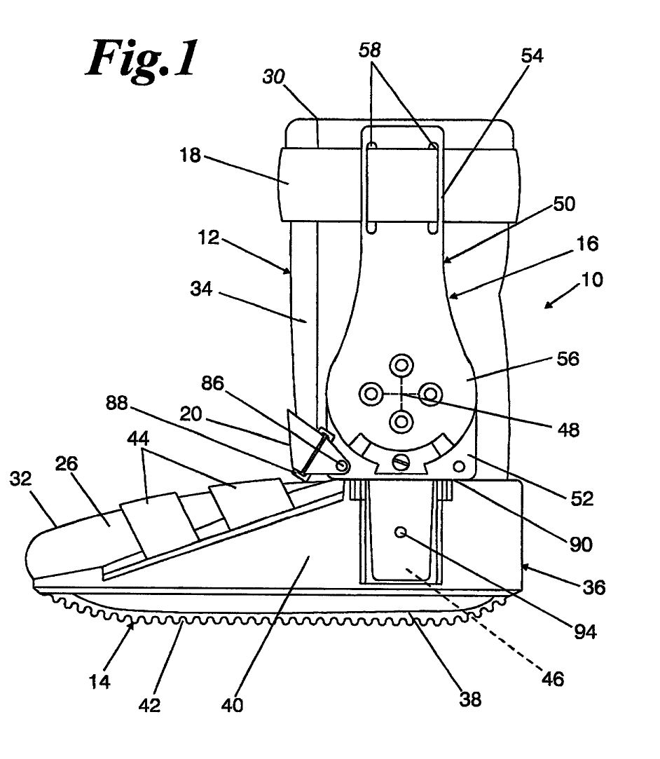

Referring to the drawings, and particularly to FIG. 1, a controlled motion ankle walker 10 is provided for controlling the range of motion of a joint, particularly an ankle joint, to reduce rehabilitation time for injuries to the joint. The ankle walker 10 includes a sleeve 12 for cushioning around the ankle joint of a patient, a rocker-type foot-supporting shoe 14 for attachment to the foot of the patient, a pair of hinges 16 (only the medial hinge is shown) extending from the shoe along the medial and lateral sides of the ankle joint and along the lower leg of the patient above the ankle joint, and upper and lower straps 18, 20 for attaching the hinges 16 to the lower leg of the patient.

The soft, flexible sleeve 12 of the ankle walker wraps around the lower leg, ankle region, and foot of the patient. The sleeve 12 can be made of one piece which includes an upper section which wraps around the circumference of the lower leg, L-shaped ankle section which wraps around the ankle region, and a foot section 26 which wraps around the foot. Preferably, the flexible sleeve 12 has a continuous front opening extending the full length of the sleeve 12 so that the front portion of the sleeve 12 can open apart continuously from the top 30 of the sleeve 12 to the end of the sleeve 12 at the foot section 26. One side of the upper section of the sleeve 12 is formed as a flexible upper flap 34 to overlap the opposite side of the sleeve 12 to form a closure extending along the front upper portion of the sleeve 12. Similarly, one side of the foot section 26 of the sleeve 12 is formed as a flexible lower flap to form a closure extending along the top of the foot section 26 of the sleeve 12. A plurality of spaced apart Velcro®-type hook and loop means (not shown) are secured to the inside of the flaps for mating to the Velcro® sensitive outerface of the sleeve 12 for snugly holding the sleeve 12 around the lower, ankle region, and foot of the patient.

The rocker-type shoe 14 can be of any suitable configuration. The preferred rocker-type shoe 14 includes a rigid open top shell 36 which is generally U-shaped in cross-section to form a bottom surface 38 with lateral and medial sidewalls 40 extending above the bottom surface 38. A rugged slip resistant sole 42 is secured to the curved bottom surface 38 of the shell 36. The inside of the shoe 14 has a rigid foot supporting interior surface (not shown) spaced above the curved bottom surface of the shoe 14. A foot pad (not shown) is normally placed on top of the rigid foot-supporting surface. A pair of flexible straps 44 are attached to the medial side wall 40 of the shoe 14 and arranged to extend over the top of the patients foot for engagement with D-rings (not shown) attached to the lateral side wall of the shoe 14. The straps 44 preferably have complementary sections of hook and loop materials, such as for example Velcro®, so that the straps can be threaded through the D-rings, tensioned the appropriate amount, and then folded back on top of themselves so that the hook type material can be frictionally attached to the loop material to hold the set amount of tension. Lateral and medial pockets 46 are respectively formed in the lateral and medial sidewalls 40 of the shoe 14 for respectively receiving and securing the lateral and medial hinges 16 to the shoe 14.

The pair of hinges 16 are respectively positioned on medial and lateral sides of the ankle joint to provide corresponding hinged pivot points aligned in a common axis through the axis of rotation 48 of the ankle joint. The pair of hinges 16 have an upper strut 50 for extending along the medial and lateral sides of the lower leg, and a lower strut 52 for rotatably attaching the upper strut 50 to the shoe 14 so that the upper strut 50 rotates about the hinge axis of rotation 48 of the ankle joint.

The upper strut 50 has an elongate upper end 54 and enlarged disc-shaped lower end 56. The upper end 54 includes a pair of vertical slots 58 for receiving the flexible upper strap 18. The upper strap 18 is woven through the slots 58 and has opposing ends of complementary hook and loop material, such as Velcro®, so that the strap 58 can encircle the upper section 22 of the sleeve 12, tensioned to the lower leg the appropriate amount, and then the opposing ends frictionally mated together to hold the set amount of tension. The Velcro® strap 18 allows the controlled motion ankle walker 10 to be quickly and easily removed from the patient.

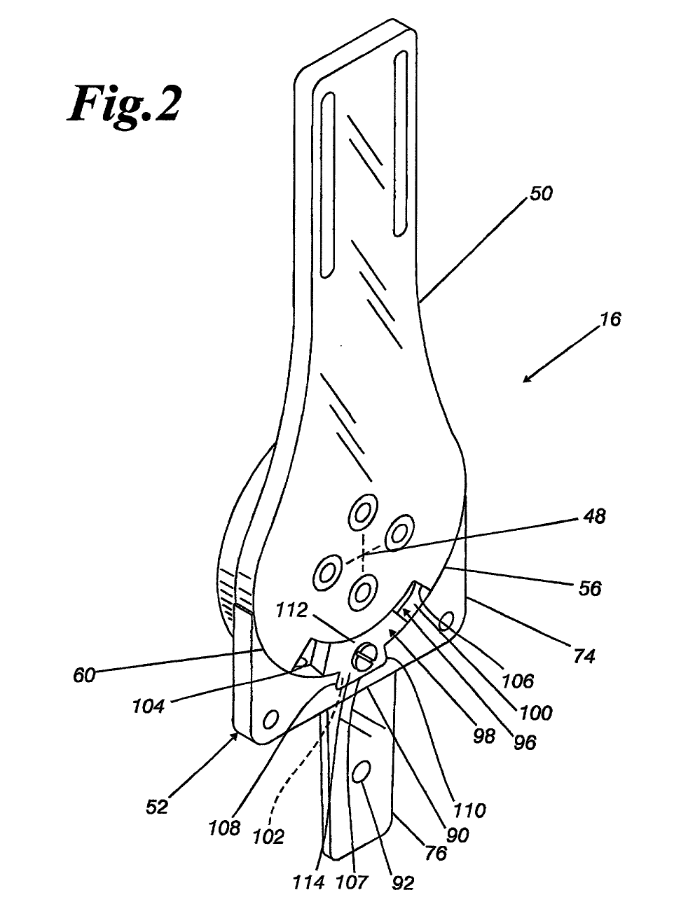

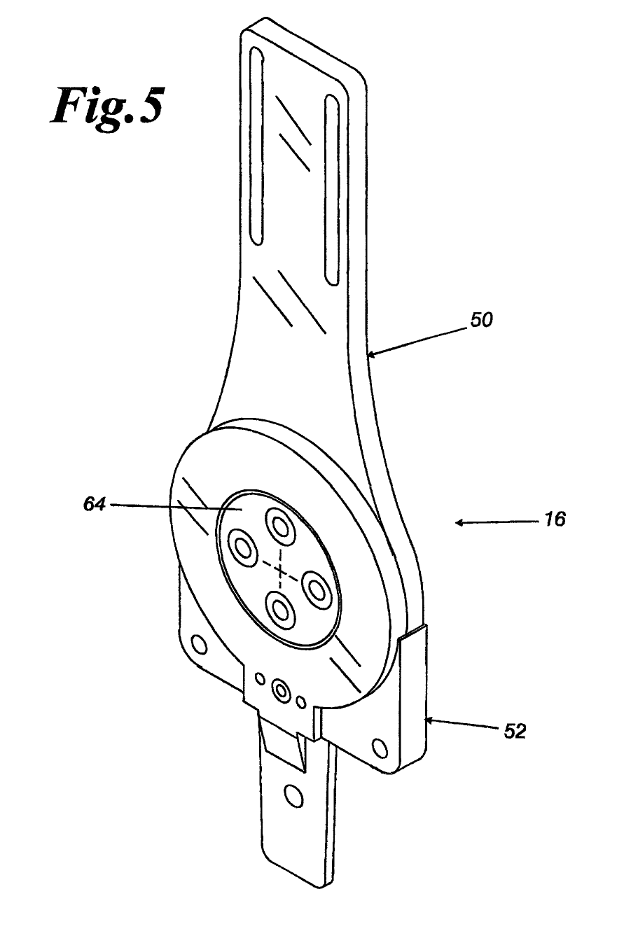

The lateral and medial hinges 16 are identical. Accordingly, the following description of the medial hinge 16 is applicable for the lateral hinge as well. FIGS. 2-6 are illustrative of the hinge 16 configuration and respectively show the hinge 16 in front perspective view, an exploded front view of the hinge 16, a perspective view of the hinge 16 with a chock 98 detached, a rear perspective view of the hinge 16, and an exploded view showing the rear of the hinge 16.

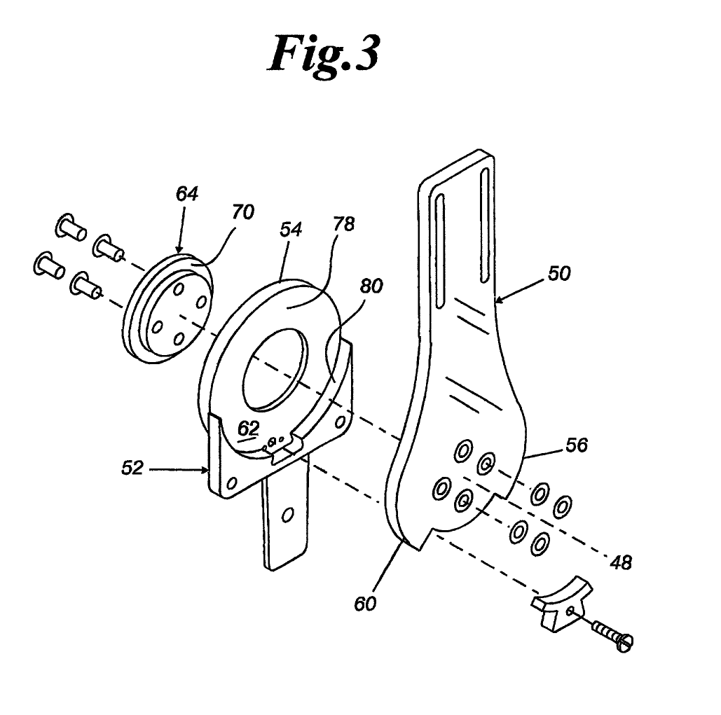

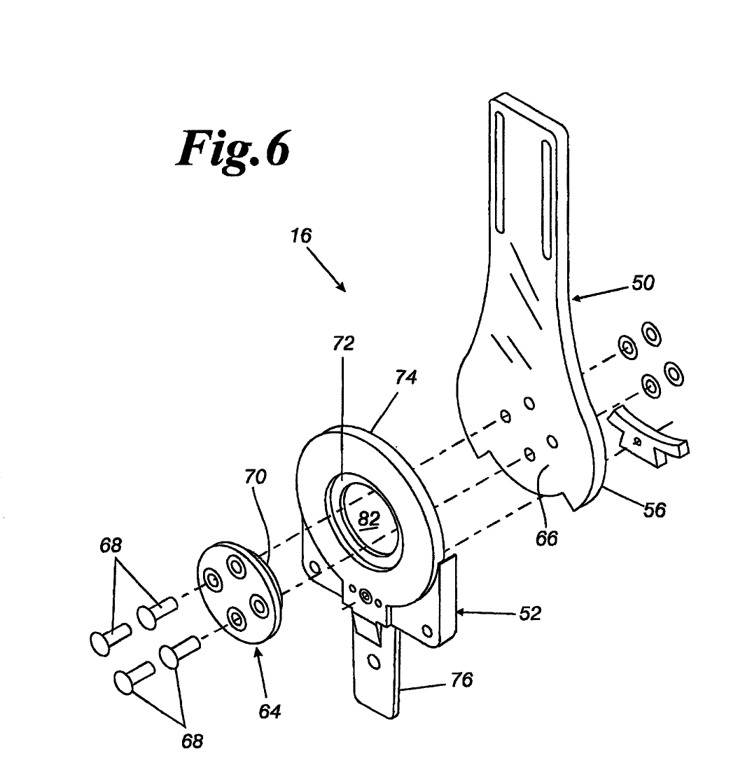

As shown in FIG. 3, the upper strut disc-shaped lower end 56 has a stepped semi-circular edge 60 extending at least 180 degrees and preferably about 270 degrees for rotating within a complementary circular recess 62 formed in the lower strut 52, and a circular enlarged axle 64 concentric with the axis of rotation 48 which defines the pivot about which the upper strut rotates with respect to the lower strut 52. As illustrated in FIG. 6, the enlarged axle 64 extends from the inside face 66 of the upper strut 50 towards the patient's ankle and is integral with the upper strut 50 by any suitable means such as, for example, the shown rivets 68, or made as one unitary part with the upper strut 50. Although the axle 64 can be made with a small diameter, preferably the enlarged axle 64 is at least one inch in diameter, and more preferably in the range of about 1¼ inches to 2 inches in diameter to distribute high force loads while providing smooth, even pivoting action. Accordingly, the hinge 16, including the upper strut 50, lower strut 52 and enlarged axle 64, need not be made of metal, but instead can be made from light-weight injection molded plastic composite materials such as, without limitation, Celcon and Delrin acetals and other strong plastic materials such as glass-filled nylon. An endless channel 70 (see also FIG. 3) is formed along the circumference of the enlarged axle 64 at its base into which an endless lip 72 from the lower strut 52 is received for rotatably attaching the lower strut 52 to the upper strut 50.

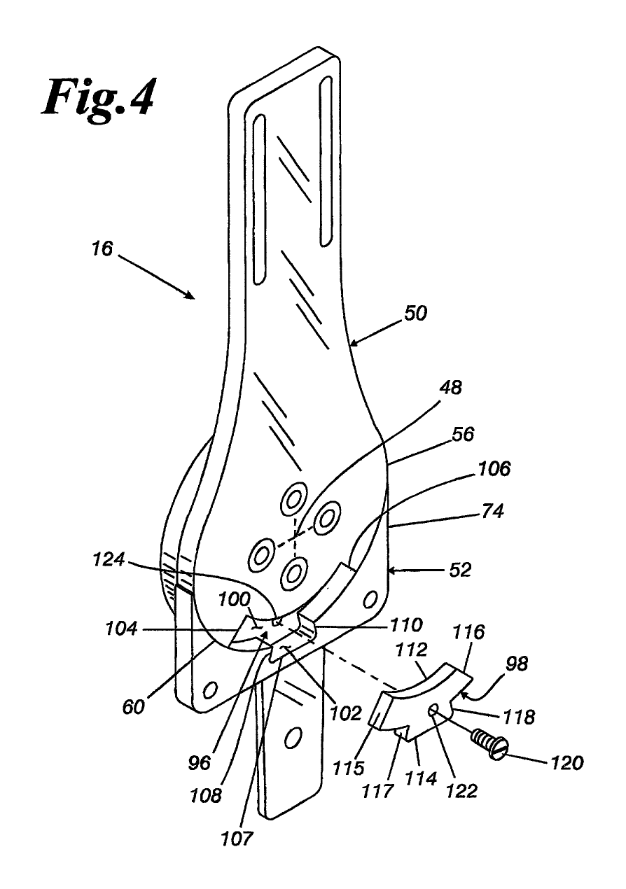

The lower strut 52 has an upper end 74 which substantially overlaps the lower end 56 of the upper strut 50, and an elongate lower end 76 for attaching the hinge 16 to the shoe 14 (FIG. 1). Referring to FIG. 3, the circular recess 62 is formed in the outward facing surface 78 of the lower strut upper end 54 and generally has the same shape and thickness as the upper strut lower end 56 that rotatably moves therein. Accordingly, the upper strut 50 nest within the recess 62 so that the front face of the hinge 16 is generally planar (See FIG. 4). A small tolerance between the upper strut circular edge 60 and a semi-circular wall 80 of the lower strut 52 allows rotation without interference.

Referring to FIG. 6, an opening 82 provided through the lower strut upper end 74 is about the same size as the enlarged axle 64. The opening 82 is framed by the endless lip 72 which is received within the axle channel 70 to rotatably and securely attach the upper strut 50 to the lower strut 52.

Illustrated in FIG. 1, attachment means 86, such as a prong or an aperture through which a pin can be inserted, is provided in the lower front corner of the lower strut 52. A strap 87 joins the attachment means 86 to a D-ring 88 from which the lower strap 20 is arranged to extend across the front of the sleeve 12 at about the height of the ankle. The free terminal end of the lower strap 20 has complementary sections of hook and loop material, such as Velcro®, so that the lower strap 20 can be threaded through the D-ring 88, tensioned to the appropriate amount and then pulled back on top of itself so that the hook type material engages the loop material to hold the set amount of tension. The lower strap 20 facilitates quick and simple removal of the ankle walker 10 from the patient's foot.

Referring to FIG. 2 in conjunction with FIG. 1, the elongate lower end 76 of the lower strut 52 extends from a shoulder 90 of the upper end 74 into the pocket 46 of the shoe 14 for fixedly securing the hinge 16 to the shoe 14. The shoulder 90 provides a rigid abutment surface against which the shoe 14 rests. An aperture 92 is provided in the lower strut lower end 76 which is coaxial with an aperture in the pocket 46 through which a threaded fastener 94 is received for rigidly securing the hinge 16 and shoe 14 together. Alternative methods can be used for attaching the hinge 16 to the shoe 14. For example, the sidewalls of the shoe can include a spring-lock disposed within the pockets wherein the lower struts are adapted to be received and locked between the spring and the outer wall of the pocket to prevent removal therefrom as described in U.S. Pat. No. 4,974,583 to Freits, and incorporated herein by reference.

Referring to FIGS. 2 and 4, a channel 96 is formed in the outward facing surface of the hinge 16 into which a chock 98 is positioned for selectively adjusting the range of motion of the ankle walker 10 in dorsiflexion and plantar flexion about the axis of rotation 48 through the ankle joint. The channel 96 is formed of two contiguous sections 100, 102. The first section 100 is a cutout in the upper strut 50 along the circular edge 60 of the enlarged lower end 56. The first section extends through the upper strut 50 to expose the lower strut 52 there-behind, and preferably is annular, arcing about 90 degrees with a cross-section of about ¼ inch×⅜ inch, although other suitable shapes, sizes and spans can also be used. The opposed ends of the first channel section 100 serve as forward and rearward abutment surfaces 104, 106 against which the chock 98 engages when the upper strut 50 rotates about the axis of rotation 48.

The second channel section 102 is formed in the upper end 74 of the lower strut 52 beneath the axis of rotation 48. The second channel section 102 has the same depth as the first section 100 and is preferably trapezoidal, or rectangular, in shape. That is, the second section 102 has an elongate linear base 107 and two sides of equal length which extend at the same acute or right angle from the base to form opposed forward and rearward abutment surfaces 108, 110 against which the chock 98 is positioned. The top of the second section 102 opens into the first section 100.

Referring to FIG. 4, the chock 98 is removably attachable within the channel 96 to selectively set the range of motion for the ankle walker 10 in dorsiflexion and plantar flexion. The chock 98 has a first portion 112 which is annular in shape to fit within the first channel section 100, and a second portion 114 which snugly fills the second channel section 102. The first chock portion 112 extends posteriorly and anteriorly any variety of degrees within the first channel section 100 to selectively set the range of motion for the ankle walker 10 in dorsiflexion and plantar flexion. Forward and rearward shoulders 115, 116 of the first chock portion 112 serve as hard stops for the first channel section abutment surfaces 104, 106.

The second chock portion 114 snugly fits within the second channel section with minimal tolerance such that forward and rearward shoulders 117, 118 are positioned adjacent to the forward and rearward abutment surfaces 108, 110. Accordingly, the chock 98 resists lateral movement by impeding against the abutment surfaces 108, 110.

The channel 96 and the chock 98 cooperate together to provide hard stops in dorsiflexion and plantar flexion for the ankle walker 10 at specific selected angles. In particular, the rotation of the upper strut 50 in dorsiflexion about the pivot axis 48 causes the upper strut forward abutment surface 104 to engage the first chock portion forward shoulder 115, and the second chock portion rearward shoulder 118 to engage the second strut rearward abutment surface 110, to limit rotation of the upper strut 50 to a specific first angle. Likewise, rotation of the upper strut 50 in plantar flexion causes the upper strut rearward abutment surface 106 to engage the first chock portion rearward shoulder 116, and the second chock portion 117 forward shoulder to engage the second strut forward abutment surface 108, to limit rotation of the upper strut 50 to a specific second angle.

In theory, forces applied by the upper strut abutment surfaces 104, 106 to the respective first chock portion shoulders 115, 116 are transferred through the chock 98 and substantially applied from the second chock portion shoulders 117, 118 to the respective second channel section abutment surfaces 108, 110. Since the chock 98 is wedged between the upper and lower struts 50, 52, and distributes forces over sufficiently broad engaging surfaces, the chock 98 as well as the upper and lower struts 50, 52 can be made of light-weight injection molded plastic material. Furthermore, no other means is required to limit rotation of the upper strut 50.

Although many various suitable means can be used to releasably attach the chock 98 within the channel 96, preferably the chock 98 is attached by using a single threaded fastener 120. The threaded fastener 120 extends through an aperture 122 within the chock 98 and is received by a complementary threaded aperture 124 in the lower strut 52. It is noted that the fastener 120 holds the chock 98 in place, but is not necessary to keep the chock 98 from moving laterally with the channel 96, that is, towards or away from the abutment surfaces.

Since the channel 96 is on the outward face of the ankle walker brace 10, the channel 96 is fully accessible for inserting and removing the chock 98. Moreover, the chock 98 remains fully accessible when secured within the channel 96. Since the chock 98 is attached only to the lower strut 52, the chock 98 can be removed and replaced by another chock without detaching or removing any other component of the ankle walker 10 or hinge 16, except for the fastener 120. Accordingly, the chock 98 is replaceable by simply removing the threaded fastener 120 and chock 98 then inserting a new chock having a different range of motion. The patient can easily change chocks without the cumbersome task of disassembling or removing any other component of the ankle walker, or removing the ankle walker brace from the patient's foot. Furthermore, since the chock's face remains fully viewable when inserted within the channel 96, indicia on the chock 98, which correspond to angular rotation in dorsiflexion and plantar flexion, can be determined by the patient or medical professional at a glance.

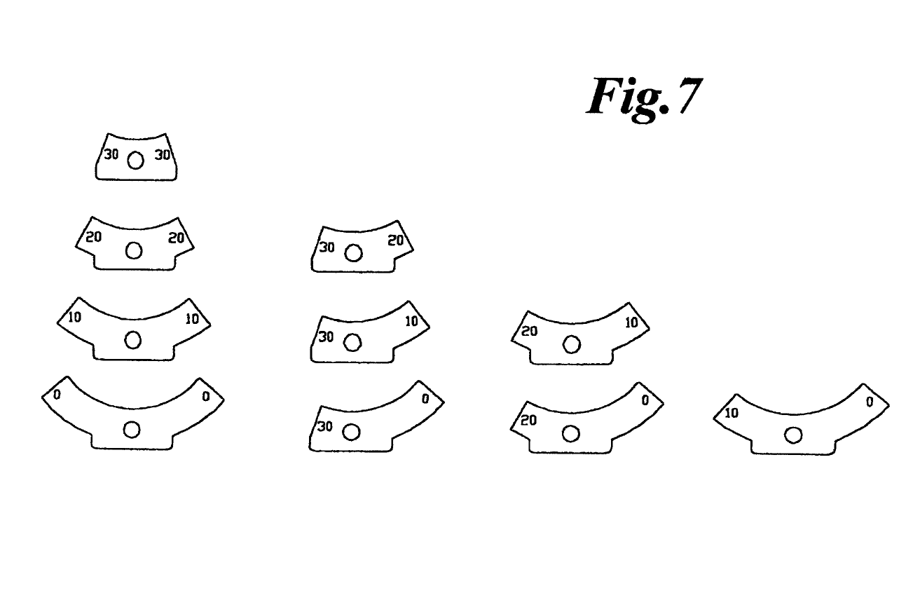

FIG. 7 illustrates a variety of chocks that can be used to provide different ranges of motion in the plantar flexion and dorsiflexion, depending on the type of rehabilitation therapy desired. Referring to FIG. 3, with no chock in place, the hinge 16 motion would range from 30 degrees in plantar flexion to 30 degrees in dorisflexion at which point the circular edge 60 of the enlarged lower end 56 of the upper strut 50 would interferingly engage with the recess wall 80 of the lower strut 52. Preferably, and not to be limiting, the chocks are pre-formed to provide a range of motion for the ankle walker from 0 to 30 degrees in plantar flexion and 0 to 30 degrees in dorsiflexion, in increments of 10 degrees. As illustrated in FIG. 4, the range of motion is selectively adjusted by inserting the desired chock within the contiguous channel 96 and securing in place by the fastener 94.

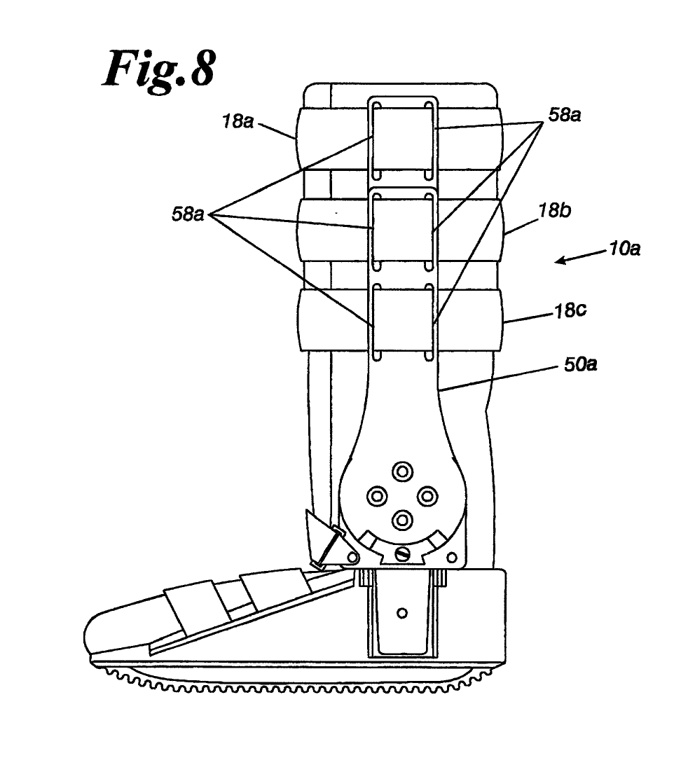

FIG. 8 shows an alternative embodiment of the invented ankle walker 10a. This embodiment is identical to the previously described ankle walker except that the upper strut 50a is further elongate to extend a greater distance along the lower leg, and has three pairs of vertical slots 58a for receiving respective straps 18a, 18b, 18c. This longer version of the ankle walker 10a is suitable for rehabilitating an injury to the tibia or fibula.

In another alternative embodiment, a variant of the hinge, similar to that disclosed for the ankle walker hinge 16, can be used to control rotational motion of a patient's knee. A sleeve is fitted and conformed to the knee region of the patient and a pair of the hinges are respectively positioned against the outer surface of the sleeve on medial and lateral sides of the knee joint. The pair of variant hinges provide corresponding hinged pivot points aligned in a common axis through the axis of rotation of the knee. The variant hinges each have an upper strut for extending above the knee and a lower strut rotatably attached to the upper strut for extending below of knee so that the upper strut rotates about the hinge axis of rotation of the knee joint.

Each upper and lower strut is each provided with at least one pair of slots, and have a sufficient length so that straps woven through the slots can properly support the hinge to the knee region. The straps can have opposing ends of complementary Velcro material to allow the hinge to be tensioned to the patient's leg, and also to allow removal.

The upper strut has a disc-shaped lower end and is rotatably joined to the lower strut as previously described for the ankle hinge 16. A contiguous channel, similar to the ankle hinge channel 96, is formed in the upper and lower struts. The channel is configured so that rotation of the alternative hinge occurs in dorsiflexion, but is restricted in plantar flexion to avoid hyperextension of the knee. A myriad of chocks are individually positionable with the channel to selectively adjust the range of motion of the variant hinge.

Referring to FIG. 1, in use the ankle walker 10 is applied to the patient's foot and lower leg by first opening the flexible padded sleeve 12 along its entire length. The sleeve 12 is closed snugly around the patient's foot and lower leg to provide a continuous flexible padding from the patient's lower leg to the foot. Flexible straps 44 are fastened over the patient's foot to further secure the patient's foot inside the shoe 14. Flexible straps 18, 20 on the hinge 16 are fastened about the patient's lower leg to apply compression to the lower leg and to hold the walker firmly in place, providing continuous support along the lower leg, ankle region, and foot.

As shown in FIG. 4, the lateral and medial hinges 16 are then set by selecting and inserting a chock 98 within the contiguous channel 96 of the hinge 16 to control the motion of the ankle walker 10 in dorisflexion and plantar flexion. The contiguous channel 96 is fully in view and accessible when the ankle walker 10 is worn by the patient. As such, the chock 98 can be positioned into, and removed from, the channel 96 without disassembling or removing any other part of the ankle walker 10, aside from the single fastener which secures the chock 98 to the lower strut 52. Moreover, since the shoulders 115, 116 of the chock first portion 112 correspond to specific first and second angles of rotation respectively in plantar flexion and dorsiflexion and do not require any adjustment, the exact range of motion is accurately set for both the lateral and medial hinges 16. When attached within the channel 96, the chock's face remains fully viewable so that the patient or medical professional can determine markings thereon which indicate the selected range of the motion.

Simply stated, the ankle walker 10 allows weight bearing through a controlled angle during rehabilitation of the ankle joint. Depending on the type of injury, the medical professional or patient can select the proper range of motion in dorsiflexion and plantar flexion to quicken the healing process. As the injured area improves, the original chock can be simply and easily replaced with another chock which allows a different range of motion for the ankle walker 10. Moreover, the injection molded plastic composite hinge 16 is much lighter than metal walkers and, accordingly, facilitates a more normal gait with less fatigue and risk of injury to the patient.

### SUMMARY OF THE ACHIEVEMENT OF THE OBJECTS OF THE INVENTION

From the foregoing, it is readily apparent that I have invented an improved controlled motion ankle walker brace for facilitating the rehabilitation process for ankle injuries and other injuries to the ankle region of the patient. In particular, the hinge of the ankle walker brace is configured to be lightweight to promote a more normal gait for the patient. Further, the range of motion in the dorsiflexion and plantar flexion can be quickly and accurately set by the patient.

It is to be understood that the foregoing description and specific embodiments are merely illustrative of the best mode of the invention and the principles thereof, and that various modifications and additions may be made to the apparatus by those skilled in the art, without departing from the spirit and scope of this invention, which is therefore understood to be limited only by the scope of the appended claims.

What is claimed:

1. A controlled motion hinge for an ankle walker brace, comprising:
a hinge including:
a first strut having a first end and a second end;
a second strut having a first end and a second end, said first end of said second strut being rotatably attached to said first strut second end so that said first strut is rotatable through an angle relative to said second strut about a pivot axis through a joint of the wearer;
a channel having:
a first section formed in said first strut second end and having forward and rearward abutment surfaces, and
a second section contiguous with said first section, formed in said second strut first end, and having forward and rearward abutment surfaces; and
a chock removably attachable within said channel, having:
a first portion having forward and rearward shoulders disposable within said first channel section, and a second portion having forward and rearward shoulders disposable within said second channel section,
so that rotation of said upper strut in a first direction about the pivot axis causes said first upper strut forward abutment surface to engage said first chock portion forward shoulder, and said second chock portion rearward shoulder to engage said second strut rearward abutment surface, to limit rotation of said first strut to a specific first angle, and
so that rotation of said first strut in a second direction about the pivot axis causes said first strut rearward abutment surface to engage said first chock portion rearward shoulder, and said second chock portion forward shoulder to engage said second strut forward abutment surface, to limit rotation of said first strut to a specific second angle.

2. The controlled motion hinge according to claim 1 wherein said second chock portion forward and rearward shoulders are respectively adjacent to said second channel section forward and rearward abutment surfaces

3. The controlled motion hinge according to claim 2 wherein force applied by said first strut forward abutment surface to said first chock portion forward shoulder is substantially transferred through said second chock portion rearward shoulder and applied to said second channel section rearward abutment surface, and wherein force applied by said first strut rearward abutment surface to said first chock portion rearward shoulder is substantially transferred through said second chock portion forward shoulder and applied to said second channel section forward abutment surface, and wherein said chock is releasably attachable to said ankle walker brace so that said chock can be replaced with another chock having a first portion having first and second shoulders at one or more different angles to limit the relative rotation between said first strut and said second strut to different first and second angles.

4. The controlled motion hinge according to claim 1 wherein said first strut and said second strut are rotatably attached by an axle and wherein said first strut, said second strut, and said axle are made of injection molded plastic composite material.

5. The controlled motion hinge according to claim 1 wherein said chock is disposed a distance from the pivot axis.

6. The controlled motion hinge according to claim 4 wherein said first strut and said second strut are rotatably attached by an axle having a diameter of at least one inch.

7. The controlled motion hinge according to claim 5 wherein said axle is integral to said first strut and has a continuous channel about a circumference of said enlarged axle and wherein said second strut has a continuous lip disposed within said continuous channel to rotatably secure said first strut to said second strut, or wherein said axle is integral to said second strut and has a continuous channel about a circumference of said axle and wherein said first strut has a continuous lip disposed within said continuous channel to rotatably secure said second strut to said first strut.

8. The controlled motion hinge according to claim 1 wherein said chock is removably attached only to said second strut.

9. The controlled motion hinge according to claim 8 further including means for removably attaching said chock within said channel.

10. The controlled motion hinge according to claim 9 wherein said means for attaching is one fastener.

11. The controlled motion hinge according to claim 9 wherein said attachment means minimally, or does not, restrict said chock from moving laterally within the channel.

12. The controlled motion hinge according to claim 10 wherein said chock is removable from said hinge and replaceable by another chock without detaching or removing any other component of the ankle walker brace or said hinge, except said fastener.

13. The controlled motion hinge according to claim 1 further including a first strap attachabed to said first end of said first strut for removably attaching said first strut to the wearer, and wherein said first end of said first strut includes at least one slot adapted for receiving said first strap.

14. The controlled motion hinge according to claim 13 further including a second strap attached to said second end of said first strut for removably attaching said first strut to the wearer.

15. The controlled motion hinge according to claim 1 wherein said first end of said second strut has an arcuate recess and wherein a majority of said second end of said first strut is rotatably disposed within said arcuate recess.

16. The controlled motion hinge according to claim 15 wherein said first end of said first strut is elongate, and said second end of said first strut is generally semi-circular and has a diameter greater than a width of said first end.

17. The controlled motion hinge according to claim 1 wherein said first channel section is shaped as a segment of an annular ring.

18. The controlled motion hinge according to claim 17 wherein said second channel section is generally shaped as a trapezoid.

19. A controlled motion hinge for an ankle walker, comprising:
a hinge including:
a first strut having a first end for extending along a side of a wearer's leg, and a second end;
a second strut having a first end rotatably attached to said first strut second end so that said first strut is rotatable through an angle relative to said second strut about a pivot axis, and a second end for attaching to a shoe;
a channel having:
a first section formed in said first strut second end; and
a second section contiguous with said first section, formed in said second strut first end;
means, releaseably attachable within said channel, for limiting rotation of said first strut in dorsiflexion and plantar flexion to respective specific angles;
means for releaseably attaching said rotation limiting means to said hinge; and
means for releaseably attaching said hinge to the lower leg of the wearer.

20. The controlled motion hinge according to claim 19 further including an axle having a diameter of at least one (1) inch which rotatably secures said first strut to said second strut.
